(19) United States
(12) Patent Application Publication
(10) Pub. No.: US 2018/0161405 A1
(43) Pub. Date: Jun. 14, 2018

(54) IMPROVED METHODS FOR TREATING OCULAR DISEASES BY GENE THERAPY

(71) Applicants: INSERM (INSTITUT NATIONAL DE LA SANTÉ ET DE LA RECHERCHE MÉDICALE), Paris Cedex 13 (FR); CENTRE HOSPITALIER UNIVERSITAIRE DE NANTES, Nantes Cedex 1 (FR); UNIVERSITÉ DE NANTES, Nantes (FR); ASSOCIATION FRANÇAISE CONTRE LES MYOPATHIES, Paris Cedex 13 (FR)

(72) Inventors: Guylène LE MEUR, Nantes (FR); Michel WEBER, Nantes (FR); Véronique BLOUIN, Sainte-Luce-sur-Loire (FR)

(21) Appl. No.: 15/577,401

(22) PCT Filed: Jun. 3, 2016
(86) PCT No.: PCT/EP2016/062659

§ 371 Date: Nov. 28, 2017

(30) Foreign Application Priority Data
Jun. 4, 2015 (EP) 15305852.4

(51) Int. Cl. A61K 38/46; A61K 48/00; A61K 9/00; A61P 27/02

(52) U.S. Cl. CPC A61K 38/465; A61K 48/005; A61K 48/0075; A61P 27/02; A61K 9/0048

(57) ABSTRACT

The invention relates to a pharmaceutical composition comprising a recombinant adeno-associated virus (r AAV) vector carrying a nucleic acid sequence encoding a functional gene under the control of regulatory sequences which express the product of said gene in the retinal cells, for use in a method for preventing or treating an inherited retinal degenerative disorder associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

### FIELD OF THE INVENTION

The invention relates to improved methods for treating ocular diseases, in particular inherited retinal degenerative disorders such as rod-cone dystrophies, with a recombinant adeno-associated virus (rAAV) carrying a nucleic acid sequence encoding a functional gene. In particular, it relates to the treatment of inherited retinal degenerative disorders by administration of said rAVV by subretinal injections in each quadrant of retina.

### BACKGROUND OF THE INVENTION

Inherited retinal degenerative disorders encompassing rod-cone dystrophies are a family of progressive diseases in which rod dysfunction, which leads to night blindness and loss of peripheral visual field expanses, is either the prevailing problem or occurring at least as severely as cone dysfunction. Rod-cone dystrophies encompass retinitis pigmentosa (RP) and Leber congenital amaurosis (LCA) including RPE65-related Leber congenital amaurosis (since more than 30 mutations in the RPE65 gene have been found to cause LCA).

Retinal pigment epithelium 65 (RPE65) is an isomerohydrolase expressed in retinal pigment epithelium and is critical for the regeneration of the visual pigment necessary for both rod and cone-mediated vision. More than 60 different mutations have been found in the RPE65 gene, accounting for approximately 2% of recessive RP cases and 16% of LCA patients. Several animal models, including the naturally occurring canine (Briard dog) model and the genetically engineered Rpe65−/− knockout mouse, have been widely used for pathological, biochemical, genetic, structural, functional and therapeutic studies ([1]).

Results in pre-clinical studies have recently led to four encouraging gene therapy clinical trials in which patients affected by LCA were sub-retinal injected with recombinant adeno-associated viral vectors rAVV2/2, containing the human RPE65 cDNA [2-6]. LCA patients have received one or two subretinal injections (mostly in superior retina in order to subserve inferior visual field function) with rAAV containing the human RPE65 cDNA. Although the purpose of these initial studies was merely to test the safety of the gene transfer agent, all 3 groups did report significant improvements in visual function. As a result of the groundbreaking positive reports, five trials (NCT00516477, NCT00643747, NCT00481546, NCT00749957, NCT 01496040 www.clinicaltrials.gov) are ongoing and expanding their patient population to examine regularly treatment safety and efficacy further. Thus, until now gene therapy for LCA is safe and effective through several years up to 6 years after vector administration [7].

These positive results provided the proof-in-principle that gene transfer ameliorates sight in visually impaired patients. However, progression of disease is not halted entirely (improvement peaking one to three years after treatment followed by a decline in visual function had been recently published) [8] so that additional improvements and/or stabilization of retinal function should be therefore obtained.

This issue prompts the researchers to the suggestion of numerous potential strategies to improve the outcome of gene therapy, including the optimization in the sequence of the RPE65 gene resulting in improved expression, the identification of the most efficient vectors, especially for expression of RPE65 in retinal cells not only in the RPE cells but also in cones, the possibility to use combination of gene therapy and other medications designed to improve the function of the visual cycle or protect the retina from loss of cells, the possibility to perform a second round of gene therapy several months or years after the first subretinal injection of AAV vector or to treat an adjacent area of the retina not yet affected by the disease, as well as the ability to stage the disease prior to treatment and guide treatment to retinal areas containing enough functional photoreceptors to respond.

### SUMMARY OF THE INVENTION

In a first aspect, the invention relates to a pharmaceutical composition comprising a recombinant adeno-associated virus (rAAV) vector carrying a nucleic acid sequence encoding a functional gene under the control of regulatory sequences which express the product of said gene in the retinal cells, for use in a method for preventing or treating an inherited retinal degenerative disorder rod-cone dystrophy associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

In a second aspect, the invention relates to a pharmaceutical composition comprising a rAAV vector carrying a nucleic acid sequence encoding a functional gene under the control of regulatory sequences which express the product of said gene in the retinal cells, for use in a method for preventing, arresting progression or ameliorating vision loss associated with an inherited retinal degenerative disorder associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

In a third aspect, the invention relates to a pharmaceutical composition comprising a rAAV vector carrying a nucleic acid sequence encoding a encoding a functional gene under the control of regulatory sequences which express the product of said gene in the retinal cells, for use in a method for enhancing retinal cell survival, including photoreceptor cell survival and retinal pigment epithelium (RPE) survival in a patient affected by an inherited retinal degenerative disorder a rod-cone dystrophy associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

### DETAILED DESCRIPTION OF THE INVENTION

The invention is based on the finding that multiple subretinal injections during the same operative period to several sites in one eye of a LCA patient (up to 5 subretinal injections), of RPE65-encoding rAAV2/4 vectors results in significant and stable morphological and functional improvement of the vision of LCA patients.

In particular, said multiple subretinal delivery of rAAV2/4-RPE65 to several sites (e.g. temporal retina, nasal retina, inferior retina and superior retina) increase the retinal surface treated by said AAV without increasing administered doses and without inducing severe retinal side effects nor leading to retinal detachment in said patient. These findings provide an improved therapeutic approach to LCA as well as other diseases associated with mutations in genes involved in inherited retinal degenerative disorders such as RP.

Accordingly, in a first aspect, the invention relates to a pharmaceutical composition comprising a recombinant adeno-associated virus (rAAV) vector carrying a nucleic acid sequence encoding a functional gene under the control of regulatory sequences which express the product of said gene in the retinal cells, for use in a method for preventing or treating an inherited retinal degenerative disorder associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

The invention also relates to a method for preventing or treating an inherited retinal degenerative disorder associated with mutations in a gene of interest comprising a step of administering in a patient in need thereof an effective amount of a pharmaceutical composition comprising a rAAV vector carrying a nucleic acid sequence encoding the functional gene of interest under the control of regulatory sequences which express the product of said gene in the retinal cells during the same operative period by at least one subretinal injection in each quadrant of retina of the patient in need thereof and wherein said quadrants consist of infero-temporal retina, supero-temporal retina, infero-nasal retina and supero-nasal retina.

As used herein, the term “rAAV vector” refers to an AAV vector carrying a nucleic acid sequence encoding a functional gene (i.e a polynucleotide of interest) for the genetic transformation of a retinal cell in a patient having a deleterious mutation in said gene. The rAAV vectors contain 5′ and 3′ adeno-associated virus inverted terminal repeats (ITRs), and the polynucleotide of interest operatively linked to sequences, which regulate its expression in a target cells, within the context of the invention, preferably or specifically in the retinal cells. Moreover, the term “rAAV vector” encompasses individual rAAV vector systems and rAAV-based dual vector systems that provide for expression of full-length proteins whose coding sequence exceeds the polynucleotide packaging capacity of individual rAAV vector. Indeed, the gene content of a rAAV vector was found to be limited to approximately 5 kB of DNA. Such rAAV dual vector systems for gene therapy of ocular diseases have been extensively described in the international patent applications no WO 2013/075008 and WO 2014/170480.

In one embodiment, the rAAV vector belongs to a AAV serotype selected in a group comprising AAV1, AAV2, AAV3, AAV4, AAV5, AAV8, AAV9, AAV10, and rhesus macaque-derived serotypes including AAVrh10, and mixtures thereof (i.e. a rAAV hybrid vector).

As used herein, the term “rAAV hybrid vector”, herein designates a vector particle comprising a native AAV capsid including an rAAV vector genome and AAV Rep proteins, wherein Cap, Rep and the ITRs of the vector genome come from at least 2 different AAV serotypes. The hybrid vector of the invention may be for instance a rAAV2/4 vector, comprising an AAV4 capsid and a rAAV genome with AAV2 ITRs or a rAAV2/5 vector, comprising an AAV5 capsid and a rAAV genome with AAV2 ITRs.

In one embodiment, said rAAV is AAV2/2, AAV2/4 serotype or AAV2/5 serotype.

A “coding sequence” is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence. Accordingly, the vector comprise regulatory sequences allowing expression and, secretion of the encoded protein, such as e.g., a promoter, enhancer, polyadenylation signal, internal ribosome entry sites (IRES), sequences encoding protein transduction domains (PTD), and the like. In this regard, the vector comprises a promoter region, operably linked to the polynucleotide of interest, to cause or improve expression of the protein in infected cells. Such a promoter may be ubiquitous, tissue-specific, strong, weak, regulated, chimeric, inducible, etc., to allow efficient and suitable (preferential) expression of the protein in the infected cells. The preferred promoters for use in the invention should be functional in retinal cells such as photoreceptor cells and retinal pigment epithelium (RPE) cells.

Examples of ubiquitous promoters include viral promoters, particularly the CMV promoter, CAG promoter (chicken beta actin promoter with CMV enhancer), the RSV promoter, the SV40 promoter, etc. and cellular promoters such as the PGK (phosphoglycerate kinase) promoter. Examples of specific promoters for retinal cells include specific promoters for RPE cells and specific promoters for photoreceptor cells. Examples of specific promoters for RPE cells are for instance the RPE65, the BEST1, the Rhodopsin the rhodopsin kinase (RK) or the cone arrestin promoters.

Examples of specific promoters for photoreceptor cells are for instance the beta phosphodiesterase gene, the retinitis pigmentosa gene promoter, the interphotoreceptor retinoid-binding protein (IRBP) gene enhancer and the IRBP gene promoters.

The rAAV vector such as the rAAV2/4 vector of the invention are produced using methods known in the art. In short, the methods generally involve (a) the introduction of the rAAV vector into a host cell, (b) the introduction of an AAV helper construct into the host cell, wherein the helper construct comprises the viral functions missing from the rAAV vector and (c) introducing a helper virus into the host cell. All functions for rAAV virion replication and packaging need to be present, to achieve replication and packaging of the rAAV vector into rAAV virions. The introduction into the host cell can be carried out using standard virological techniques simultaneously or sequentially. Finally, the host cells are cultured to produce rAAV virions and are purified using standard techniques such as CsCl gradients. Residual helper virus activity can be inactivated using known methods, such as for example heat inactivation. The purified rAAV virion is then ready for use in the methods.

As used herein, the term “patient” is intended for a human. Typically the patient is affected or likely to be affected with an inherited retinal degenerative disorder, especially rod-cone dystrophy, affecting the retinal pigment epithelium (RPE) cells or the photoreceptors cells. For instance, patients are candidates for the methods of treatment include those who have a diagnosis of LCA. Typical symptoms of LCA include: severe vision impairment from birth; nystagmus (involuntary jerky rhythmic eye movement); a normal-appearing eye upon visual examination (though there may be some pigmentation on the retina); extreme farsightedness; a slow pupillary response to light; and markedly reduced ERGs. A diagnosis of LCA can be made, e.g., based on Lambert's criteria (Lambert et al., Sury Ophthalmol. 1989; 34(3):173-86).

The methods described herein can include identifying a patient, e.g., a child, adolescent, or young adult subject with LCA or who is suspected of having LCA (e.g., based on the presence of symptoms of LCA and no other obvious cause), and obtaining a sample comprising genomic DNA from the patient, detecting the presence of a mutation in a gene known as responsible for LCA such as RPE65 using known molecular biological methods, and selecting a patient who has such a mutation that causes LCA. Detecting a mutation in a gene of interest as RPE65 can include detecting a specific known mutation.

Accordingly a wide variety of retinal diseases may thus be treated given the teachings provided herein and typically include inherited retinal degenerations in particular retinitis pigmentosa (RP) and rod-cone dystrophies such as Leber's congenital amaurosis (LCA).

In a particular embodiment, said rod-cone dystrophy is Leber congenital amaurosis (LCA). Originally described by Leber in 1869, LCA is an autosomal recessive disease distinct from other retinal dystrophies and responsible for congenital blindness. Leber congenital amaurosis (LCA) (MIM 204000) is characterized by severe or complete loss of visual function apparent early in infancy with failure to follow visual stimuli, nystagmus, and roving eye movements. Affected individuals have an extinguished electroretinogram and eventually develop abnormalities of the ocular fundus including a pigmentary retinopathy. LCA is a severe childhood-onset blinding disease which may be caused by mutations in more than 10 genes. The most frequently mutated genes are CEP290, GUCY2D, CRB1 and RPE65. Accordingly, more than 10 types of LCA are recognized as described in the Table below:

| Type | OMIM | Gene | Locus |
|---|---|---|---|
| LCA1 | 204000 | GUCY2D | 17p13.1 |
| LCA2 | 204100 | RPE65 | 1p31 |
| LCA3 | 604232 | RDH12 | 14q23.3 |
| LCA4 | 604393 | AIPL1 | 17p13.1 |
| LCA5 | 604537 | LCA5 | 6q11-6q16 |
| LCA6 | 605446 | RPGRIP1 | 14q11 |
| LCA7 | 602225 | CRX | 19q13.3 |
| LCA8 | 604210 | CRB1 | 1q31-q32.1 |
| LCA9 | 608553 | NMNAT1 | 1p36 |
| LCA10 | 610142 | CEP290 | 12q21.33 |
| LCA11 | 146690 | IMPDH1 | 7q31.3-q32 |

In a particular embodiment, the LCA is RPE65-related LCA.

In another embodiment, said inherited retinal degenerative disorder is selected from the group consisting of retinitis pigmentosa, choroideremia, and Usher disease.

Accordingly, the nucleic acid sequence encoding a functional gene is a polynucleotide encoding a polypeptide will enhance the survival and/or function of retinal cells such as photoreceptor cells and RPE cells. Examples of polynucleotides of interest that can be used for gene replacement therapy are genes that are preferentially or specifically expressed in photoreceptor cells and/or RPE cells, such as RPE65 (LCA, chr. 1), RGR (Retinitis pigmentosa (RP), chr. 10), RLBP1 (RP, chr. 15), MERTK (RP, chr. 2), LRAT (RP, chr. 4), REP1 (choroideremia, Xp21), MYO7A (Usher syndrome type 1, chr. 11) and CEP290 (LCA, chr. 12).

The recombinant AAV vector containing the desired transgene as detailed above is preferably assessed for contamination by conventional methods and then formulated into a pharmaceutical composition intended for subretinal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. The precise nature of the carrier or other material may be determined by the skilled person according to the route of administration, i.e. here the subretinal injection. Such materials should be non-toxic and should not interfere with the efficacy of the active ingredient (i.e. the rAAV vector of the invention).

Furthermore, in certain embodiments of the invention it is desirable to perform non-invasive retinal imaging and functional studies to identify areas of retained photoreceptors to be targeted for therapy. In these embodiments, clinical diagnostic tests are employed, to determine the precise location(s) for one or more subretinal injection(s). These tests may include electroretinography (ERG), perimetry, topographical mapping of the layers of the retina and measurement of the thickness of its layers by means of confocal scanning laser ophthalmoscopy (eSLO) and optical coherence tomography (OCT), topographical mapping of cone density via adaptive optics (AO), functional eye exam, etc.

In view of the imaging and functional studies, the volume and viral titer of each injection is determined individually, as further described below, and may be the same or different from other injections performed in the same, or contralateral, eye.

By “effective amount” is meant an amount sufficient to achieve a concentration of rAAV composition which is capable of preventing, treating or slowing down the disease to be treated. Such concentrations can be routinely determined by those of skilled in the art. The amount of the rAAV composition actually administered will typically be determined by a physician, in the light of the relevant circumstances, including the disease to be treated, the chosen route of administration, the age, weight, and response of the patient, the severity of the patient's symptoms, and the like. It will also be appreciated by those of skilled in the art that the dosage may be dependent on the stability of the administered rAAV vector.

In one embodiment, the volume and concentration of the rAAV composition is selected so that only the region of damaged photoreceptors is impacted. In another embodiment, the volume and/or concentration of the rAAV composition is a greater amount, in order reach larger portions of the eye, including non-damaged photoreceptors.

The pharmaceutical composition may be delivered in a volume of from about 50 μL to about 1 mL, including all numbers within the range, depending on the size of the area to be treated, the viral titer and the desired effect of the method. In one embodiment, the volume is about 50 μL. In another embodiment, the volume is about 100 μL. In another embodiment, the volume is about 150 μL. In yet another embodiment, the volume is about 200 μL. In another embodiment, the volume is about 250 μL. In another embodiment, the volume is about 300 μL. In another embodiment, the volume is about 400 μL. In another embodiment, the volume is about 450 μL. In another embodiment, the volume is about 500 μL. In another embodiment, the volume is about 600 μL. In another embodiment, the volume is about 750 μL. In another embodiment, the volume is about 800 μL. In another embodiment, the volume is about 900 μL. In yet another embodiment, the volume is about 1000 μL.

The doses of vectors may be adapted depending on the disease condition, the patient, the treatment schedule, etc. A preferred effective dose within the context of this invention is a dose allowing an optimal transduction of the photoreceptors and/or RPE cells. Typically, from 10⁸ to 10¹⁰ viral genomes (vg) are administered per dose in mice. Typically, the doses of AAV vectors to be administered in humans may range from 10¹⁰ to 10¹² vg.

Accordingly, an effective concentration of a recombinant adeno-associated virus carrying a nucleic acid sequence encoding the desired transgene desirably ranges between about 10⁸ and 10¹³ vector genomes per milliliter (vg/mL). The rAAV infectious units are measured as described in S. K. McLaughlin et al, 1988 J. Virol, 62: 15*63. Preferably, the concentration is from about 1×10⁹ vg/mL to about 1×10¹² vg/mL, and more preferably from about 1×10¹⁰ vg/mL to about 1×10¹¹ vg/mL. In one embodiment, the effective concentration is about 5×10¹⁰ vg/mL.

Still other dosages in these ranges may be selected by the attending physician, taking into account the physical state of the patient, being treated, the age of the subject, the particular ocular disorder and the degree to which the disorder, if progressive, has developed.

For each of the described methods, the treatment may be used to prevent the occurrence of retinal damage or to rescue eyes having mild or advanced disease.

As used herein, the term “rescue” means to prevent progression of the disease to total blindness, prevent spread of damage to uninjured photoreceptor cells and/or RPE cells or to improve damage in injured photoreceptor cells and/or RPE cells.

Thus, in one embodiment, the pharmaceutical composition is administered before disease onset. In another embodiment, the pharmaceutical composition is administered prior to the initiation of photoreceptor loss. In another embodiment, the pharmaceutical composition is administered after initiation of photoreceptor loss. In yet another embodiment, the pharmaceutical composition is administered when less than 90% of the photoreceptors are functioning or remaining, as compared to a non-diseased eye. In another embodiment, the pharmaceutical composition is administered when less than 50% of the photoreceptors are functioning or remaining. In another embodiment, the pharmaceutical composition is administered when less than 40% of the photoreceptors are functioning or remaining. In another embodiment, the pharmaceutical composition is administered when less than 30% of the photoreceptors are functioning or remaining. In another embodiment, the pharmaceutical composition is administered when less than 20% of the photoreceptors are functioning or remaining. In another embodiment, the pharmaceutical composition is administered when less than 10% of the photoreceptors are functioning or remaining.

As used herein, the term “same operative period” refers to the period that begins when the patient is transferred to the operating room bed and ends with the transfer of a patient to the postanesthesia care unit (PACU). During this period the patient is monitored, anesthetized, prepped, and draped, and the operation is performed. Nursing activities during this period focus on safety, infection prevention, and physiological response to anesthesia. The term “same operative period” is thus meant that the multiple injections may be performed simultaneously or sequentially (at different time points and with equal or different time intervals).

Each retina to be treated is divided into quadrants. Accordingly, the surface of the retina is subdivided by vertical and horizontal lines that intersect at the center of the fovea. The vertical line divides the retina into nasal and temporal divisions and the horizontal line divides the retina into superior and inferior divisions. Corresponding vertical and horizontal lines in visual space (also called meridians) intersect at the point of fixation (the point in visual space that the fovea is aligned with) and define the quadrants of the visual field. Thus, the retina comprises four quadrants consisting of infero-temporal retina, supero-temporal retina, infero-nasal retina and supero-nasal retina.

In one embodiment, at least one subretinal injection is performed in each quadrant of retina. In another embodiment, two subretinal injections are performed in at least one quadrant of retina. In another embodiment, two subretinal injections are performed in each quadrant of retina. In another embodiment, three subretinal injections are performed in at least one quadrant of retina. In another embodiment, three subretinal injections are performed in each quadrant of retina.

Thus, the pharmaceutical composition may be formulated in a large volume such as about 750 μL or 800 μL and is delivered in several times in each quadrant of the retina. Alternatively, the pharmaceutical composition may be formulated in a small volume and is delivered in one time in one quadrant of the retina. In such a case, several units are required.

Subretinal injections may be performed or not under general anesthesia.

Moreover, subretinal injections may be performed by virtue of a device for liquid micro-injection in confined medium such as an eye as described in the international patent application n° WO 03/094992. Such device for liquid micro-injection comprises at least one plunger-type syringe bearing a small diameter injection cannula, means for driving the plunger for injection, control means for the plunger driving means, the driving means being of the pneumatic type controlled by a mobile member capable of being actuated by an operator. The driving means comprise a pressurized gas which acts directly on the plunger and means supplying pressurized gas into the syringe upon contact with the plunger. The control means comprise a mobile member capable of being actuated by an operator to apply gas pressure to the syringe plunger and to cancel said pressure.

In a second aspect, the invention relates to a pharmaceutical composition comprising a rAAV vector carrying a nucleic acid sequence encoding a functional gene under the control of regulatory sequences which express the product of said gene in the retinal cells, for use in a method for preventing, arresting progression or ameliorating vision loss associated with an inherited retinal degenerative disorder associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

The invention also relates to a method for preventing, arresting progression or ameliorating vision loss associated with an inherited retinal degenerative disorder with mutations in a gene of interest comprising a step of administering in a patient in need thereof an effective amount of a pharmaceutical composition comprising a rAAV vector carrying a nucleic acid sequence encoding the functional gene of interest under the control of regulatory sequences which express the product of said gene in the retinal cells during the same operative period by at least one subretinal injection in each quadrant of retina of the patient and wherein said quadrants consist of infero-temporal retina, supero-temporal retina, infero-nasal retina and supero-nasal retina.

As used herein, the term “vision loss” associated with rod-cone dystrophy refers to any decrease in peripheral vision, central (reading) vision, night vision, day vision, loss of color perception, loss of contrast sensitivity, or reduction in visual acuity.

In a third aspect, the invention relates to a pharmaceutical composition comprising a rAAV vector carrying a nucleic acid sequence encoding a encoding a functional gene under the control of regulatory sequences which express the product of said gene in the retinal cells, for use in a method for enhancing retinal cell survival, including photoreceptor cell survival and retinal pigment epithelium (RPE) survival in a patient affected by an inherited retinal degenerative disorder associated with mutations in said gene, wherein the pharmaceutical composition is administered during the same operative period by at least one subretinal injection in each quadrant of retina of the patient in need thereof and wherein said quadrants consist of infero-temporal retina, supero-temporal retina, infero-nasal retina and supero-nasal retina.

The invention also relates to a method for enhancing retinal cell survival, including photoreceptor cell survival and RPE survival in a patient affected by an inherited retinal degenerative disorder associated with mutations in a gene of interest comprising a step of administering in said patient an effective amount of a pharmaceutical composition comprising a rAAV vector carrying a nucleic acid sequence encoding the functional gene of interest under the control of regulatory sequences which express the product of said gene in the retinal cells during the same operative period by at least one subretinal injection in each quadrant of retina of the patient and wherein said quadrants consist of infero-temporal retina, supero-temporal retina, infero-nasal retina and supero-nasal retina.

As used herein, the term “enhancing retinal cell survival”, including photoreceptor cell survival and retinal pigment epithelia survival, is meant as inhibiting or slowing degeneration of a retinal cell, and increasing retinal cell viability, which can result in slowing or halting the progression of an ocular disease or disorder or retinal injury, described herein.

In one embodiment, the retinal cell is a photoreceptor cell and/or a retinal pigmental epithelium (RPE).

The invention will be further illustrated by the following figures and examples. However, these examples and figures should not be interpreted in any way as limiting the scope of the present invention.

### FIGURES

FIG. 1: Inflammation evaluation after subretinal injection of rAAV2/4.hrpe65.rpe65. (A) Graph of the laser flare meter measure at D-90, D-1, D+4, D+14, D+60, D+180, D+360. (B) Value of the laser flare meter for the three patients who present a modification of the value at D+4 and D+14 in Ph/ms.

FIG. 2: Sensorial and oculomotricity evaluation. (A) ETDRS visual acuity results in injected eye and uninjected eye before injection and at the last visit one year post injection, presence of nystagmus, presence of exotropia. (B) Variation of the visual acuity mean after the surgery in the untreated eye and in the treated for the nine patients. (C) Variation of the visual acuity mean after the surgery in the untreated eye and in the treated for the nystagmic patients. LP: light perception; TE: treated eye; UE, untreated eye.

FIG. 3: Follow up of visual field based on injected surface. (A) For the three columns: on the left a composite photograph of patient retina; the area exposed to the vector is note with the dashed line, on the right the goldmann visual field; in clear, V4 surface before injection, in dark the V4 surface at one year post injection. (B) Variation of the mean visual field surface after the surgery for treated eye in dark and for untreated eye in bright for night patients. (C) Variation of the visual field average gain based on the injected dose of the vector.

### EXAMPLE: SAFETY AND EFFICACY OF GENE TRANSFER WITH A AAV4 FOR RPE65 LEBER'S CONGENITAL AMAUROSIS

Material & Methods

The Trial:

This clinical trial (NCT01496040) is a Phase I/II study that was approved by the Tours-Ouest 1 Ethics Committee on 4 Mar. 2011 and by Afssaps on 1 Sep. 2011. After information had been given to the patient or legal guardian, consent to participate was obtained. Patients were divided into three groups according to the dose of virus injected and their age. Adult patients in the first group were given the lowest dose of viral vector (up to 400 μl) and the other two groups were given higher doses, up to 800 μl of solution for adults in the second group and children in the third. An independent safety data monitoring board appointed to monitor the study was convened between patients 1 and 2, 3 and 4, 4 and 5, 6 and 7, 7 and 8 to gauge the safety and tolerance of AAV2/4.rpe65.rpe65.

Patients:

Patients included in this study all carried two mutations in the rpe65 gene (checked at baseline) (Table 1):

TABLE 1
Demographic and genetic characteristics of the patients.

| | Age | DNA allele 1 | Protein 1 | DNA allele 2 | Protein 2 |
|---|---|---|---|---|---|
| CG01 | 28 Y | c.700C > T | p.Arg234X | c.1067delA | Asn356Methfs*17 |
| BJ03 | 27 Y | c.544C > G | p.His182Asp | c.726 − 2A > G | fs* |
| MM04 | 35 Y | c.444G > C | p.Glu148Asp | c.1451G > A | p.Gly484Asp |
| MR05 | 42 Y | c.74C > T | p.Pro25Leu | c.1301C > A | p.Ala434Glu |
| HM06 | 22 Y | c.843_858 + 7del23 | p.Asn282fs* | c.843_858 + 7del23 | p.Asn282fs* |
| HT07 | 20 Y | 440_441delCA | p.Thr147Argfs*9 | c.1448_1450delATG | p.Asp483del |
| AM08 | 19 Y | 246 − 11A > G | fs* | c.615_616delCA | p.Ile206Cysfs*27 |
| HM09 | 15 Y | c.989 G > A | p.Cys 330 Tyr | c.843_858 + 7del23 | p.Cys 330 Tyr |
| LC10 | 9 Y | c.11 + 5G > A | fs* | c.1039C > T | p.Arg347Cys |

DNA, desoxynucleotidic acid;
Y, year

Vector Production:

The pAAV-hRPE65 vector plasmid carries the transgene expression cassette flanked by AAV serotype 2 inverted terminal repeats (ITRs). The expression cassette contains the human RPE65 coding sequence (NCBI RefSeq NM_000329) under control of a human RPE65 promoter fragment (positions −1359 to +23 relative to the transcription start site), and a bovine growth hormone polyadenylation signal.

For production of the rAAV-2/4.hRPE65 vector, pAAV-hRPE65 plasmid was transfected into HEK293 cells together with pDP4-Kana helper plasmid, which provides both AAV serotype 4 rep and cap genes and adenovirus helper genes (VA RNA, E2A and E4). The vector was purified by ion-exchange chromatography and formulated in a saline solution specific for ocular surgery.

The rAAV-2/4.hRPE65 vector was filled in 0.5 mL aliquots into 1.2 mL cryovials. Concentration of the final drug product was 6×10¹⁰ vector genomes per mL, as titered by dot blot hybridization.

Surgery and Peroperative Treatment:

Sub-retinal injection was performed under general anesthetic into the eye with the worst visual function. Vitrectomy (20 gauge, three channels) was performed before injection using a 41G cannula. The patient was kept still for 20 minutes after the surgery to promote contact between the viral vector and EPR cells.

Since retinal detachment varied from one patient to the next, different volumes were injected into each patient, between 200 μl and 800 μl, corresponding to 1.22. 10¹⁰-4,8. 10¹⁰ vector genomes (Table 2). The number of sub-retinal injection sites was between two and four in each operation (Table 2) with the sites chosen to favor treatment of the peripheral, extramacular retina.

TABLE 2
Patients injection characteristics.

| | | Vol | Vector genom | Injection number |
|---|---|---|---|---|
| First cohort | CG01 | 330 μL | 2.01 · 10¹⁰ | 2 |
| | BJ03 | 200 μL | 1.22 · 10¹⁰ | 3 |
| | MM04 | 300 μL | 1.83 · 10¹⁰ | 3 |
| Second cohort | MR05 | 700 μL | 4.27 · 10¹⁰ | 5 |
| | HM06 | 770 μL | 4.7 · 10¹⁰ | 2 |
| | HT07 | 530 μL | 3.23 · 10¹⁰ | 4 |
| Third cohort | AM08 | 700 μL | 4.27 · 10¹⁰ | 4 |
| | HM09 | 800 μL | 4.8 · 10¹⁰ | 4 |
| | LC10 | 770 μL | 4.7 · 10¹⁰ | 3 |

Vol, volume;
μL, microliters

A week after injection, patients were given oral prednisolone (½ mg/kg/day) then 1 mg/kg/day for a week after surgery. This dose was then stepped down over the next month. Topical postoperative treatment consisted of dexamethasone-tobramycin eye drops (three times a day for a month) together with 1% atropine eye drops in the operated eye (once daily for seven days).

Assessment of Dissemination of the Viral Vector:

After surgery, patients were kept in a confinement chamber from D0 to D+3. Biodissemination of the AAV2/4.RPE 65 vector was analyzed in serum, nasal discharge and urine before injection of the viral vector and then one, two and three days after injection. Tests were carried out by qPCR PREMIX EX TAQ (Perfect Real Time) TAKARA (Sigma), Fluo: FAM/TAMRA (Eurogentec). The Kit QlAamp Viral RNA mini kit (QIAGEN) was used for extraction: 1 cycle at 95° C. for 10 minutes, 45×15-second cycles at 95° C., 45×30-second cycles at 62° C. The limit of detection was 25 copies and the limit of quantitation was 100 copies.

Safety:

A routine ophthalmologic examination was carried out with microscopic inspection of the anterior chamber and vitreal cavity. Retinal inflammation was scored on the Nussenblatt scale combined with a Tyndall protein measurement in the anterior chamber using a laser flare meter (Kowa FM700). Chorioretinal tolerance was assessed on photographs of the retina according to the ETDRS method using non-mydriatic retinography (TOPCON TRC-NW6S) after dilatation of the pupil (tropicamide, Ciba Vision Faure, Novartis, Annonay, France). Macular thickness, retinal structure and nerve fibre thickness were analyzed by spectral domain OCT (Heidelberg Engineering, Spectralis HRA-OCT). The thickness of the external nuclear layer was measured manually by two different observers at the fovea then at points 300 μm and 1000 temporal and nasal to the fovea (Heidelberg Engineering, Spectralis HRA-OCT). Angiography (Heidelberg Engineering, Spectralis HRA-OCT) with fluorescein (5 mL fluorescein sodium) and Indocyanin Green (Infracyanine®, SERB) was carried out to observe vascular and retinal changes following vector injection. Physical examinations, blood chemistry and hematological tests were carried out before and after sub-retinal injection.

Patients filled in a safety questionnaire on eye pain, ocular discomfort and blurred vision after surgery.

An Immunological Study:

Humoral Responses to AAV4 Vector: The analyses were performed in INSERM 1089 laboratory under the control of our quality management system that is approved by Lloyd's Register Quality Assurance LRQA to meet requirements of international Management System Standards ISO 9001:2008.

The detection of anti-AAV4 IgG antibodies in patient sera was performed using an Enzyme Linked Immuno Sorbent Assay (ELISA) with a method validated according to the ICH(Q2 R1) quality guideline. Briefly, patient sera were serially diluted in PBS-Tween 0.1% buffer and incubated in 96 well plates pre-coated with recombinant AAV2/4 viral particles. The reaction was revealed after incubation with peroxidase conjugated donkey anti-human IgG F(ab′)2 fragment (Jackson Immunoresearch), and TMB substrate (BD Biosciences). Optical densities were read (450 nm-570 nm) using a microplate spectrophotometer reader (MultScan GO, Thermo). For each dilution, the threshold of positivity was determined as the mean of optic densities+3SD obtained independently with 19 negative serum from healthy donors. For positive samples, IgG titer was defined as the last serum dilution with an optical density remaining above the threshold curve.

Neutralizing factors against AAV4 were detected using a neutralization assay. The assay is based on the inhibition of Cos cell line transduction in the presence of serial serum dilutions using an AAV4 vector expressing the Green Fluorescent Protein (GFP) reporter gene. Percentages of GFP positive cells were determined by flow cytometry 72 hours after cell infection. The neutralizing titer was defined as the highest serum dilution inhibiting the AAV transduction by ≥50% in comparison with the transduction control without serum.

Cellular immune responses to AAV4 vector and RPE65 transgene product: Cellular immune responses against AAV4 capsid and RPE65 gene product were evaluated using IFNγ ELISpot assays, and were performed at the immunology platform of Nantes University Hospital and when necessary, for some sample second runs, at INSERM 1089 laboratory. Briefly, frozen PBMC were plated in anti-INFγ precoated 96-well ELIspot plates (human INFγ ELISpot plus kit, Mabtech) and stimulated in the presence of an overlapping peptide library at the final concentration of 2 μg/ml (Pepscreen, Sigma) covering either the sequence of AAV4 VP1 capsid protein (divided in 3 pools), or the sequence of RPE65 protein (divided in 2 pools). The reaction was revealed 24 hours after cell stimulation according to the manufacturer instruction (human INFγ ELISpot plus kit, Mabtech). The results were expressed as spot-forming units (SFC)/10⁶ cells. A positive response to any peptide pool was arbitrarily defined as a SFC/10⁶ response >50 SFC/10⁶ cells and at least 3 times higher than the number of spots recorded with non-activated cells (medium alone).

Efficacy:

Distant visual acuity was scored on the ETDRS scale and near visual acuity on the Parinaud scale. Color perception was assessed with a monocular, saturated 15-hue test. When visual acuity was better than 20/200, changes in visual field were assessed using an automatic perimeter visual field (Octopus 101 perimeter, Haag-streit Inc, Koeninz, Switzerland) coupled to semi-static Goldmann analysis in V4. Visual field areas were analyzed using Allplan 2015 software with statistical analysis by R software (Version 3.0, R Foundation for Statistical Computing, Vienna, Austria). Microperimetry with a 4-2 strategy was carried out after 10 minutes of dark adaptation using 200 ms stimuli u to a luminescence of 127 cd/m² (Nidek MP1 microperimeter-NAVIS software version 1.7.1, Nidek Technologies, Padova, Italy). Broad-field ERG according to the ISCEV protocol was carried out on a vision monitor (Monpack3, Metrovision, Perenchies, France). When fixation was good enough, multifocal ERG was carried out on a RETIscan system (Roland Consult, Wiesbaden, Germany) with RETIscan software (version 3.15) in line with ISCEV recommendations. Dynamic pupillometry was used to measure pupil size and rates of dilatation and contraction in response to a series of flashes was measured using a Vision Monitor Pupillometry device (Metrovision, Perenchies, France). In order to assess changes in patients' capacity for displacement after sub-retinal injection, a mobility test was carried out. The displacement time for patients with either the operated eye or the other one covered up was measured in milliseconds. For this test, patients had to move round a maze with two different light levels (4 lux and 240 lux) with the path chosen randomly. The test was repeated in triplicate for each eye and in each lighting condition. A questionnaire about the patients' impressions of their vision was administered after surgery.

Functional MRI:

The inventors use a block design study, one run consisting in three 30 second conditions presented alternatively 4 times:
condition 1: rest in darkness, without any visual stimulation.
condition 2: white uniform screen flickering (5 Hz). Luminance will be constant during the 30 second presentation, but will be modified from low to high level between the 4 repetitions.
condition 3: black and white full screen checkerboard flickering (5 Hz). Luminance will be constant during all the presentation, but the checkerboard contrast will be modified from low to high level between the 4 repetitions.

Each subject will undergo 3 runs during the fMRI session. Comparing recorded activities between conditions 1 and 2 will show cortical responses to luminance modulations; Activity between conditions 1 and 3 will be related to contrast modulations.

Visual stimulations will be generated with specific software to control images luminance and contrast. Functional acquisitions will be made with a 1.5 Tesla Magnetic Resonance system and a standard head coil. Functional data will be acquired with T2*-weighted gradient-Echo Planar Image (EPI) sequences. T1 weighted three-dimensional anatomical acquisitions (MP-RAGE) will be recorded at the end of the session. Individual MRI data will be analyzed with SPMS software package (Wellcome Department of Cognitive Neurology, London, U.K.).

Results

The patients were between 15 and 42 years of age at the time of surgery (Table 1). All carried mutations in the rpe65 gene. Since retinal detachment varied from one patient to the next, different volumes were injected into each patient, between 200 μl and 800 μl, corresponding to 1.22. 10¹⁰-4.8. 10¹⁰ vector genomes (Table 2). The number of sub-retinal injection sites was between two and four in each operation (Table 2) with the sites chosen according to either the residual visual field prior to the operation or peroperative retinal detachment.

During the year of follow-up, no systemic adverse effects were reported following sub-retinal injection of the AAV2/4-Rpe65-Rpe65 vector in any of the nine treated patients. Pre- and post-operative ocular inflammation was measured with a Laser Flare meter.

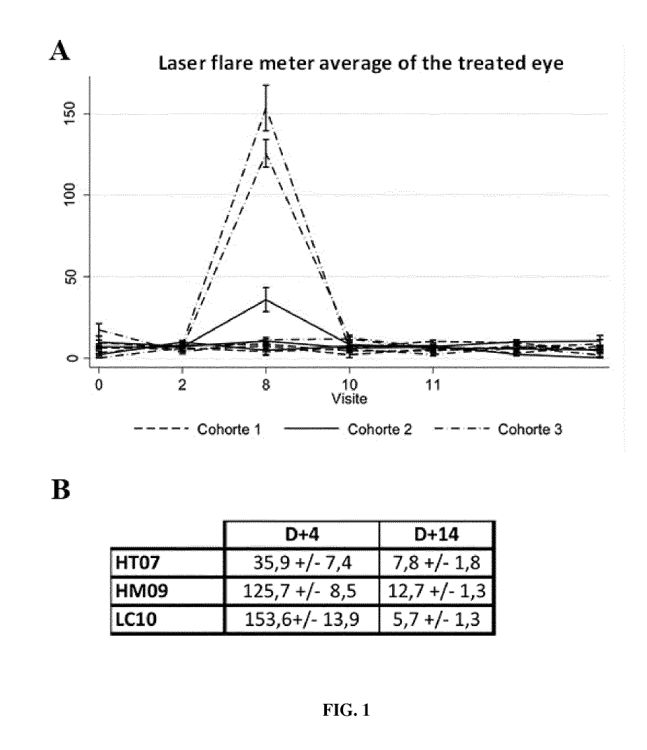

Increased inflammation was observed on D+4 in three patients (HT07, HM09 and LC10) with return to normal 14 days after sub-retinal injection (FIG. 1A). For patient HT07, topical anti-inflammatory treatment was stepped up to six instillations a day from D+3 and continued throughout the hospital stay, resulting in rapid regression of the inflammation. More severe inflammation was observed in the other two patients with a Laser Flare readings of 125.7±8.5 ph/ms and 153.6±13.9 ph/ms on D+4 followed by normalization by D+14 (FIG. 1B). These two patients received doses of 800 μL and 770 μL of viral vector suspension and sub-retinal injection bubbles had been observed during the operation, especially in the vitreous with a bubble that spread out somewhat over the surface. For patient LC10, the bubble took over four days to disappear with a sheet of vector fluid still visible in the OCT examination performed four days after injection.

Ophthalmologic monitoring did not detect any adverse effects during the year of follow-up, i.e. no retinal detachment or cataract. No adverse systemic effects were reported with no changes in hematological parameters or blood chemistry results at a series of different time points. The safety questionnaire revealed some itching and pain at the suture points immediately after surgery and lasting a few days. Angiography did not detect postoperative inflammatory or vascular abnormalities. The only significant facts were a mask effect at the spots where the cannula had been inserted into the retina for the injection, which left a scar.

In the distribution analysis, the viral vector was mostly detected in postoperative samples of nasal discharge. In four patients (BJ03, HM06, HT07 and LC10), between 4 and 201 copies were measured with peak leaching of the viral vector around D+2. Only in BJ03 and LC10 were viral load readings above the limit of detection and, in LC10 above the limit of quantitation with a peak of 204 copies measured in the tears on D+2. Between D0 and D+2, virus was only detected in the blood of one patient, HM06: this was temporary and low-level (24 and 19 copies). No virus was ever detected in urine. Patients were able to leave the confinement chamber on D+3.

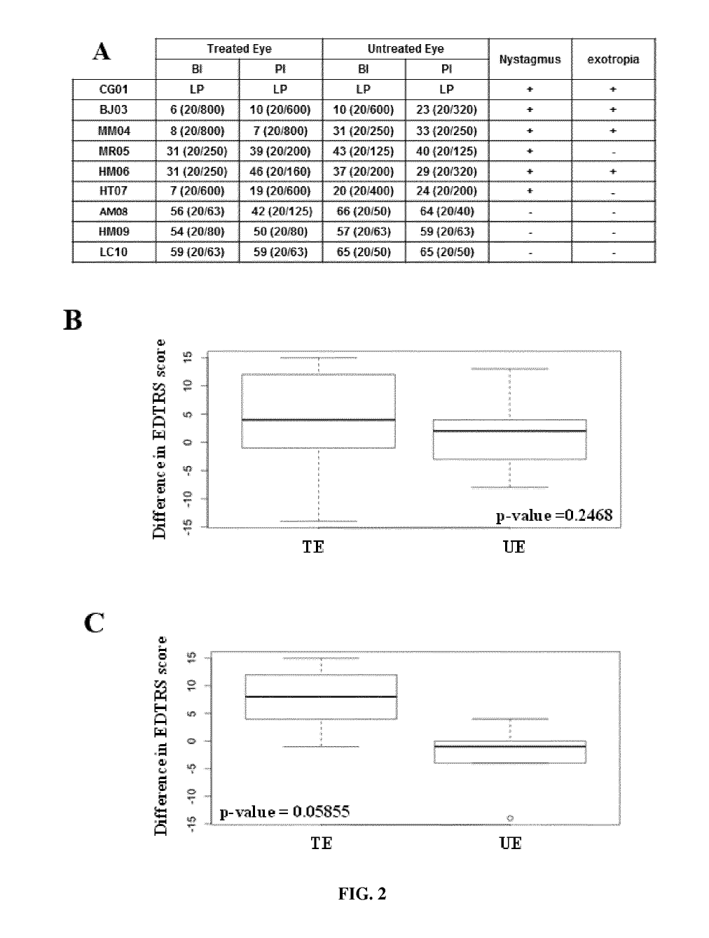

Six of the nine included patients had nystagmus (FIG. 2A) and four of them have divergent strabismus (FIG. 2A). Patient HM06 saw his director eye change with preferential fixation with the treated eye following surgery. Patient HT07 reported preferring to use his treated eye which had originally been the weaker one for near vision with installation of alternating fixation according to distance. MR05 reported a change in sensation of modification of ocular dominance following surgery. Visual acuity increased by 2.5 EDTRS letters after surgery in the treated eye and by 1 EDTRS letter in the other one (FIG. 2B). There was a difference in visual acuity gain between patients with and without nystagmus. In those with nystagmus, the gain was +7.6 EDTRS letters in the treated eye and +1.6 letters in the other one (FIG. 2C). This difference approaches significance (p=0.05855). Patients HT07 and HM08 showed the strongest gain in visual acuity (+15 and +12 EDTRS letters).

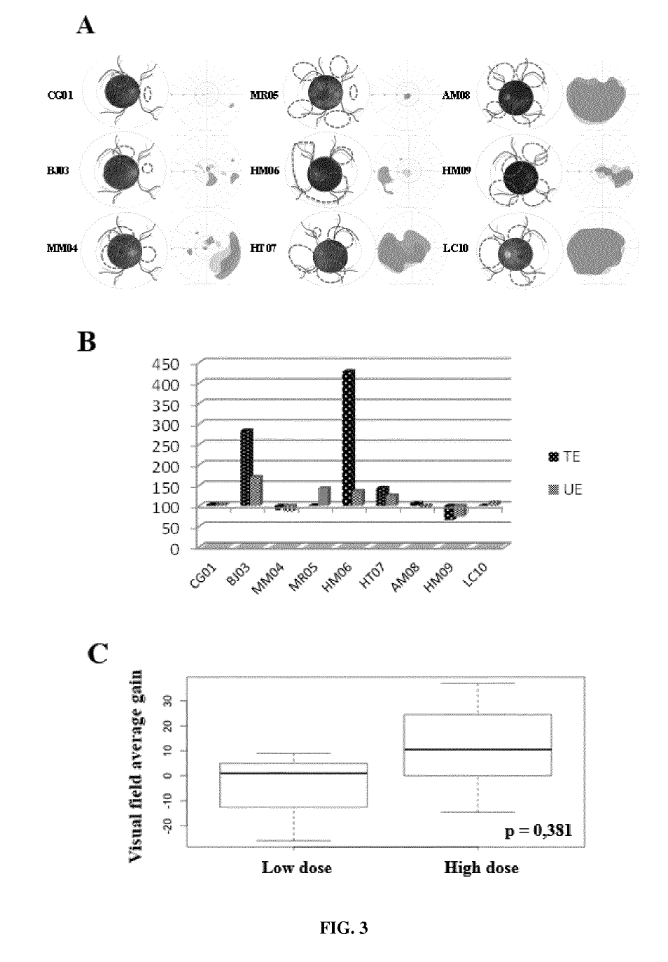

Change in visual field varied from one subject to the next. It improved in patients CG01, BJ03, HM06, HT07, AM08 and LC01, it remained unchanged in MR05 (the oldest patient) and it decreased in MM04 and HM09 (FIGS. 3A and 3B). Some patients like HM06 and BJ03 saw the gain in visual field area multiplied respectively by factors of 4.2 and 2.8 (FIG. 3C). In contrast, visual field shrunk in MM04 and HM09 by respective factors of 0.9 and 0.65 (FIG. 3C). Visual field recovery was greatest in patients injected with the highest dose with a mean loss of 5.32667 in patients injected with the lowest dose compared with a mean gain of 11.293167 in those injected with the highest dose. Recovery seems to correlate with the dose of vector injected although the result for this small sample is insignificant (p=0.381).

No change in electroretinographic pattern was observed after sub-retinal injection of the AAV2/4.rpe65 vector.

The efficacy questionnaire revealed improved detail perception in four out of nine patients, improved fixation in three and, in one patient each, improved color vision, reduced photophobia and less visual fatigue.

### Discussion

The safety of sub-retinal injection of the retinal epithelium-specific AAV2/4-Rpe65-Rpe65 vector was evaluated in patients with Leber's congenital amaurosis due to a defective rpe65 gene. No adverse systemic or ophthalmologic effects were reported in any of the nine patients treated.

Several sub-retinal injections with 2-4 retinotomies in the course of this study did not lead to any adverse effects in the retina. No retinal detachment was observed immediately after surgery or in one year of follow-up. Multiple injections mean that a greater retinal surface area can be treated, adapted to the preoperative state of the retina. Monitoring of postoperative ocular inflammation showed that some patients experience transient and moderate inflammation as measured using a Flare Meter. This increase was observed in three patients injected with the highest dose of vector in the D+4 examination but not on D+14. In these patients, we had observed that the sub-retinal injection bubble was dominant in the vitreous during surgery with slower disappearance (over 24 hours). It is likely that diffusion of the vector into the vitreous happens some time after injection which meant that we saw peak inflammation after four days. Nevertheless, vitrectomy alone without viral vector injection induces a rise in Laser Flare reading with a peak within a week of surgery, e.g. vitrectomy for rhegmatogenous retinal detachment (Hoshi) and all the more so because patients with pigmentary retinopathy have a modified hematoretinal barrier (Murikami).

### REFERENCES

Throughout this application, various references describe the state of the art to which this invention pertains. The disclosures of these references are hereby incorporated by reference into the present disclosure.
1. Cai X, Conley S M, Naash M I. RPE65: role in the visual cycle, human retinal disease, and gene therapy. Ophthalmic Genet. 2009; 30(2):57-62.
2. Bainbridge J W, Smith A J, Barker S S, Robbie S, Henderson R, Balaggan K, Viswanathan A, Holder G E, Stockman A, Tyler N, Petersen-Jones S, Bhattacharya S S, Thrasher A J, Fitzke F W, Carter B J, Rubin G S, Moore A T, Ali R R. Effect of gene therapy on visual function in Leber's congenital amaurosis. N. Engl. J. Med. 2008; 358(21):2231-2239.
3. Cideciyan A V, Aleman T S, Boye S L, Schwartz S B, Kaushal S, Roman A J, Pang J J, Sumaroka A, Windsor E A, Wilson J M, Flotte T R, Fishman G A, Heon E, Stone E M, Byrne B J, Jacobson S G, Hauswirth W W. Human gene therapy for RPE65 isomerase deficiency activates the retinoid cycle of vision but with slow rod kinetics. Proc. Natl. Acad. Sci. USA. 2008; 105(39):15112-15117.
4. Maguire A M, Simonelli F, Pierce E A, Pugh E N Jr, Mingozzi F, Bennicelli J, Banfi S, Marshall K A, Testa F, Surace E M, Rossi S, Lyubarsky A, Arruda V R, Konkle B, Stone E, Sun J, Jacobs J, Dell'Osso L, Hertle R, Ma J X, Redmond T M, Zhu X, Hauck B, Zelenaia O, Shindler K S, Maguire M G, Wright J F, Volpe N J, McDonnell J W, Auricchio A, High K A, Bennett J. Safety and efficacy of gene transfer for Leber's congenital amaurosis. N. Engl. J. Med. 2008; 358(21):2240-2248.
5. Maguire A M, High K A, Auricchio A, Wright J F, Pierce E A, Testa F, Mingozzi F, Bennicelli J L, Ying G S, Rossi S, Fulton A, Marshall K A, Banfi S, Chung D C, Morgan J I, Hauck B, Zelenaia O, Zhu X, Raffini L, Coppieters F, De Baere E, Shindler K S, Volpe N J, Surace E M, Acerra C, Lyubarsky A, Redmond T M, Stone E, Sun J, McDonnell J W, Leroy B P, Simonelli F, Bennett J. Age-dependent effects of RPE65 gene therapy for Leber's congenital amaurosis: a phase 1 dose-escalation trial. Lancet. 2009 Nov. 7; 374(9701):1597-605.
6. Simonelli F, Maguire A M, Testa F, Pierce E A, Mingozzi F, Bennicelli J L, Rossi S, Marshall K, Banfi S, Surace E M, Sun J, Redmond T M, Zhu X, Shindler K S, Ying G S, Ziviello C, Acerra C, Wright J F, McDonnell J W, High K A, Bennett J, Auricchio A. Gene therapy for Leber's congenital amaurosis is safe and effective through 1.5 years after vector administration. Mol Ther. 2010 March; 18(3):643-50.
7. Jacobson S G, Cideciyan A V, Ratnakaram R, Heon E, Schwartz S B, Roman A J, Peden M C, Aleman T S, Boye S L, Sumaroka A, Conlon T J, Calcedo R, Pang J J, Erger K E, Olivares M B, Mullins C L, Swider M, Kaushal S, Feuer W J, Iannaccone A, Fishman G A, Stone E M, Byrne B J, Hauswirth W W. Gene therapy for leber congenital amaurosis caused by RPE65 mutations: safety and efficacy in 15 children and adults followed up to 3 years. Arch Ophthalmol. 2012 January; 130(1):9-24.
8. Jacobson S G, Cideciyan A V, Roman A J, Sumaroka A, Schwartz S B, Heon E, Hauswirth W W. Improvement and Decline in Vision with Gene Therapy in Childhood Blindness. N Engl J Med. 2015 May 3.

1-15. (canceled)

16. A method for preventing or treating an inherited retinal degenerative disorder associated with mutations in a gene in a patient in need thereof, the method comprising administering to the patient a pharmaceutical composition comprising a recombinant adeno-associated virus (rAAV) vector carrying a nucleic acid sequence encoding the functional gene under the control of regulatory sequences which express the product of said gene in the retinal cells, wherein the pharmaceutical composition is administered during the same operative period by at least one subretinal injection in each quadrant of retina of the patient, and wherein said quadrants consist of infero-temporal retina, supero-temporal retina, infero-nasal retina and supero-nasal retina.

17. The method according to claim 16, wherein preventing or treating an inherited retinal degenerative disorder comprises preventing, arresting progression or ameliorating vision loss associated with the inherited retinal degenerative disorder associated with mutations in said gene.

18. The method according to claim 16, wherein preventing or treating an inherited retinal degenerative disorder comprises enhancing retinal cell survival, including photoreceptor cell survival and retinal pigment epithelium (RPE) survival.

19. The method according to claim 16, wherein said inherited retinal degenerative disorder is retinitis pigmentosa (RP).

20. The method according to claim 16, wherein said inherited retinal degenerative disorder is Leber congenital amaurosis (LCA).

21. The method according to claim 16, wherein said functional gene is RLBP1 or RPE65.

22. The method according to claim 16, wherein said rAAV is AAV2/5 or AAV2/4 serotype.

23. The method according to claim 16, wherein the retinal cells in which the functional gene is expressed are RPE cells.

24. The method according to claim 16, wherein the pharmaceutical composition is administered before disease onset.

25. The method according to claim 16, wherein the pharmaceutical composition is administered after initiation of photoreceptor loss.

26. The method according to claim 16, wherein the pharmaceutical composition is administered when less than 50% of photoreceptors are functioning or remaining.

27. The method according to claim 16, wherein the pharmaceutical composition is administered at a concentration between 10⁹ and 10¹² vector genomes per milliliter (vg/mL).

28. The method according to claim 16, wherein the pharmaceutical composition is administered at a concentration of about 5.10¹⁰ vg/mL.

29. The method according to claim 16, wherein the pharmaceutical composition is administered in a volume of 450 μL.

30. The method according to claim 16, wherein the pharmaceutical composition is administered in a volume of 750 μL or 800 μL.
